(19) United States
(12) Patent Application Publication
(10) Pub. No.: US 2016/0151085 A1
(43) Pub. Date: Jun. 2, 2016

(54) Biopsy Tool

(71) Applicant: Donna McDonald, Rogersville, TN (US)

(72) Inventor: Donna McDonald, Rogersville, TN (US)

(21) Appl. No.: 14/954,350

(22) Filed: Nov. 30, 2015

Related U.S. Application Data
(60) Provisional application No. 62/085,374, filed on Nov. 28, 2014.

(51) Int. Cl. A61B 17/3205

(52) U.S. Cl. CPC A61B 17/32056; A61B 2017/32006

(57) ABSTRACT

A biopsy tool for removing a tissue sample from a patient. The biopsy tool includes an elongated shaft having a hollow interior volume, an open first end, and an open second end. The second end includes a cutting blade thereon for cutting into tissue. A cutting wire is disposed within the hollow interior volume and includes a loop thereon positioned adjacent to the second end of the elongated shaft and cutting blade. The loop can be used to cut the base or underside of the tissue sample in order to remove the tissue sample from the user. The movement of the cutting wire is restricted by a support positioned on the interior of the second end of the elongated shaft. A portion of the cutting wire extends outward from the first end of the elongated shaft and can be pulled in order to use the loop to cut through tissue.

### CROSS REFERENCE TO RELATED APPLICATION

This application claims the benefit of U.S. Provisional Application No. 62/085,374 filed on Nov. 28, 2014. The above identified patent application is herein incorporated by reference in its entirety to provide continuity of disclosure.

### BACKGROUND OF THE INVENTION

### Field of the Invention

The present invention relates to biopsy tools. More specifically, the present invention provides a biopsy tool comprising a cutting wire for helping to remove a tissue sample from a user without the need to utilize other tools such as forceps, surgical scissors, and/or scalpels.

It is often necessary for a doctor or other medical professional to take a sample of skin or tissue in order to examine the sample more closely. Taking a sample is often required when the patient has a lesion, growth, mass, among other abnormal tissue. The sample can then be further examined and analyzed in order for the doctor to diagnose the illness and determine the extent of the illness.

A biopsy is performed in order to collect a tissue sample for analysis. Biopsies can be performed using any of various instruments or methods, however, a punch biopsy is a common method for collecting the sample. The punch biopsy is performed using a circular blade disposed on an elongated handle which is rotated through the layers of the skin. However, the punch biopsy may cause pain and bleeding, and may not be able to collect tissue samples from various parts of the user's body. Further, the doctor must then use forceps and either scissors or a scalpel in order to fully remove the tissue sample. This can be inconvenient for the doctor and may cause the patient to experience further pain.

Devices have been disclosed in the prior art that relate to biopsy tools and cutting implements. These include devices that have been patented and published in patent application publications. These devices generally relate to biopsy tools having various configurations, such as U.S. Published Patent Application Number 2007/0093727, U.S. Published Patent Application Number 2007/0249960, U.S. Published Patent Application Number 2011/0275955, U.S. Pat. No. 6,626,903, U.S. Published Patent Application Number 2013/0211403, and U.S. Pat. No. 6,786,913.

These prior art devices have several known drawbacks. The devices in the prior art provide biopsy tools, but fail to provide a biopsy tool having a wire cutting implement wherein the wire cutting implement can be adjusted so as to cut a tissue sample from a desired location.

In light of the devices disclosed in the prior art, it is submitted that the present invention substantially diverges in design elements from the prior art and consequently it is clear that there is a need in the art for an improvement to existing biopsy tools. In this regard the instant invention substantially fulfills these needs.

### SUMMARY OF THE INVENTION

In view of the foregoing disadvantages inherent in the known types of biopsy tools now present in the prior art, the present invention provides a new biopsy tool wherein the same can be utilized for providing convenience for the user when taking a tissue sample from a patient, particularly when taking the sample from the cervical tissue of the patient.

It is therefore an object of the present invention to provide a new and improved biopsy tool comprising an elongated shaft having a cutting wire extending therethrough, wherein the cutting wire is adapted to cut a section of tissue and remove the same from the user's body.

It is another object of the present invention to provide a biopsy tool comprising a cutting wire, wherein a proximal end of the wire is formed into a circular loop.

Another object of the present invention is to provide a biopsy tool comprising a cutting wire that can be pulled by the user in order to cut the tissue of a user in order to remove a sample of the user's tissue.

Yet another object of the present invention is to provide a biopsy tool comprising a circular cutting blade disposed on the end of an elongated shaft.

Another object of the present invention is to provide a biopsy tool that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the biopsy tool.

FIG. 2 shows a transparent view of the biopsy tool.

FIG. 3A shows a close-up view of the cutting wire of the biopsy tool in a first position.

FIG. 3B shows a close-up view of the cutting wire of the biopsy tool in a second position.

FIG. 4 shows a cut out view of the cutting blade of the biopsy tool.

FIG. 5 shows a cross sectional view of the cutting blade of the biopsy tool.

### DETAILED DESCRIPTION OF THE INVENTION

Reference is made herein to the attached drawings. Like reference numerals are used throughout the drawings to depict like or similar elements of the biopsy tool. For the purposes of presenting a brief and clear description of the present invention, the preferred embodiment will be discussed as used for taking a tissue sample from a user. The figures are intended for representative purposes only and should not be considered to be limiting in any respect.

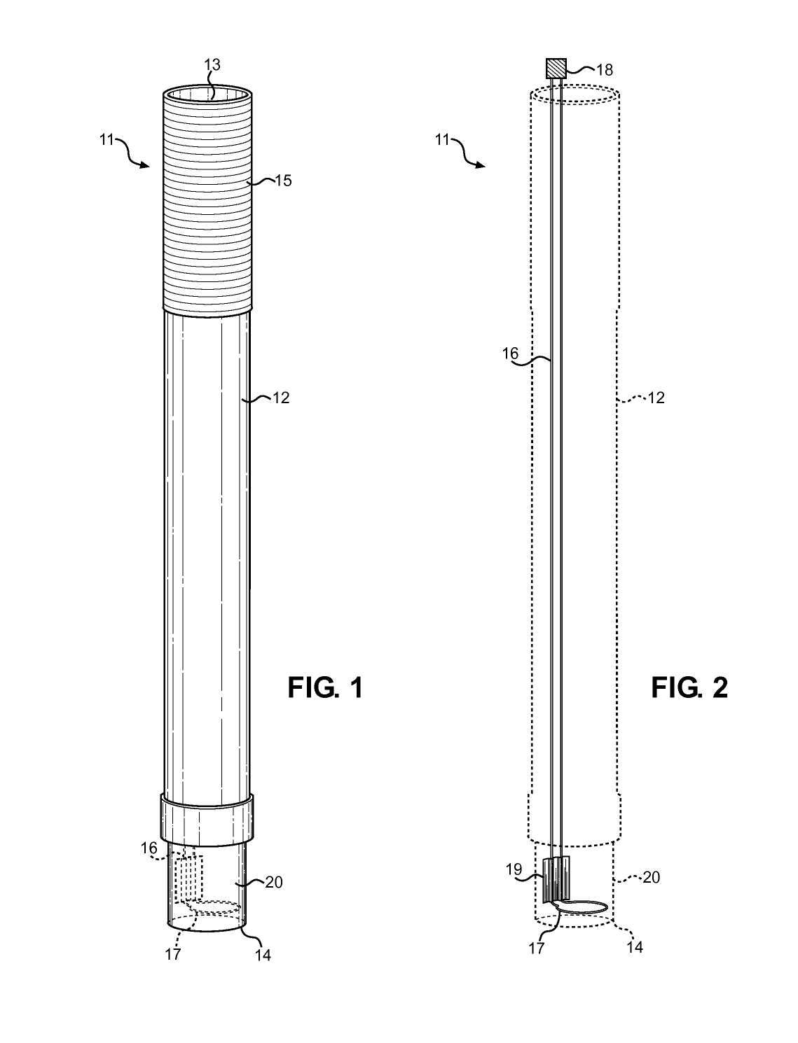

Referring now to FIGS. 1 and 2, there are shown views of the biopsy tool. The biopsy tool 11 comprises an elongated shaft 12 having a first end 13 and a second end 14. The elongated shaft 12 is preferably linear in configuration. More preferably, the elongated shaft 12 is cylindrical in configuration, however, in alternate embodiments the elongated shaft 12 can have other configurations, such as a square shaped cross sectional area. The elongated shaft 12 is preferably composed of a corrosion resistant stainless steel, or surgical steel.

The elongated shaft 12 comprises a hollow interior volume, an open first end 13 and an open second end 14. The first end 13 comprises a threaded exterior 15 adapted to removably engage with another device, such as a speculum or the like. In this way, the user can utilize the speculum to hold open a body orifice, while simultaneously utilizing the biopsy tool 11 to collect a tissue sample from the orifice. This is particularly helpful in allowing a medical professional to more easily collect a tissue sample from the cervix, such as when the medical professional is checking for cervical cancer.

The second end 14 of the elongated shaft 12 comprises a cutting blade 20 thereon. In the illustrated embodiment, the cutting blade 20 comprises a substantially circular configuration and extends from the second end 14 of the elongated shaft 12. In alternate embodiments, the cutting blade 20 may have an elliptical configuration. The cutting blade 20 is composed of a sharpened material suitable for making an incision into the tissue of a user.

The biopsy tool 11 further comprises a cutting wire 16 positioned within the hollow interior volume of the elongated shaft 12. The cutting wire 16 extends from the first end 13 of the elongated shaft 12 to the second end 14 thereof. The cutting wire 16 is formed into a loop 17 at the second end 14 thereof. Preferably, the loop 17 comprises a circular shape and is similar in shape to the cutting blade 20. The loop 17 is oriented perpendicularly relative to the longitudinal axis of the elongated shaft 12.

The cutting wire 16 extends through a support cuff 19 disposed on the interior of the second end 14 of the elongated shaft 12. The support cuff 19 comprises a tubular member that helps to restrain and limit the movement of the cutting wire 16. The diameter of the loop 17 can be manually adjusted by the user by pulling the portion of the cutting wire 16 at the first end 13 of the elongated shaft 12, which draws a portion of the loop 17 through the support cuff 19, reducing the diameter or size of the loop 17. The cutting wire 16 preferably comprises a grip 18 adjacent to the first end of the elongated shaft 12 adapted to allow a user to more easily pull the cutting wire 16. As the cutting wire 16 is pulled, the diameter of the loop 17 decreases, so that the cutting wire 17 can sever the base or underside of a tissue sample, allowing the tissue sample to be removed from the body of the user without the use of forceps, surgical scissors, and/or a scalpel.

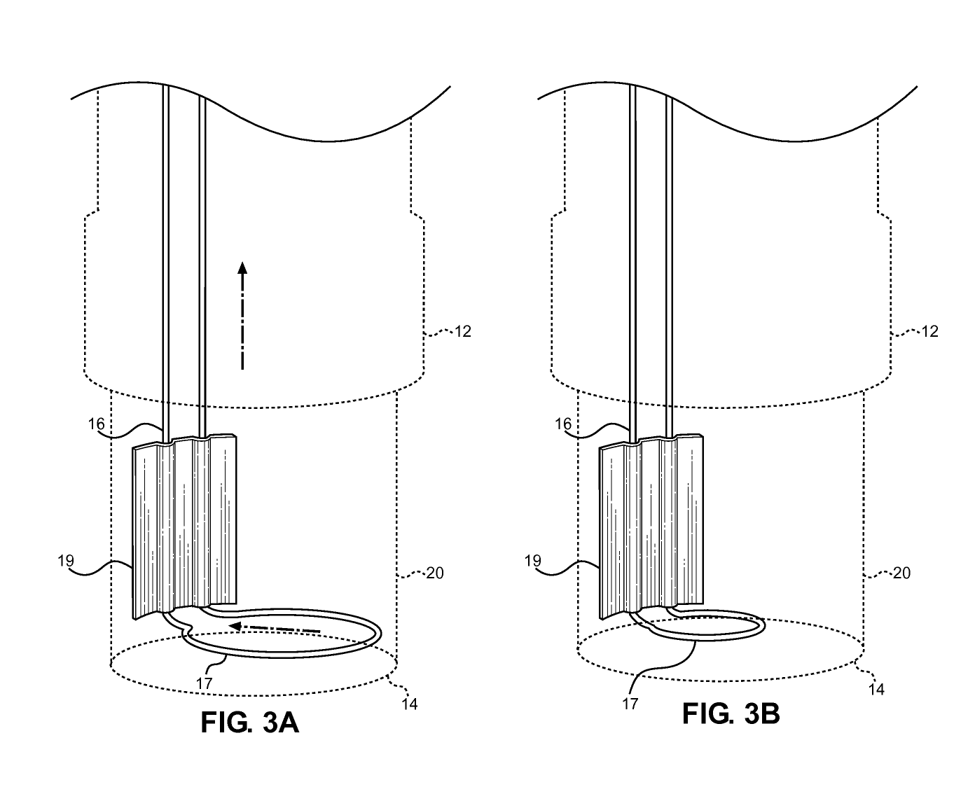

Referring now to FIGS. 3A and 3B, there is shown a close-up view of the cutting wire of the biopsy tool. In operation, the user can hold the shaft 12 of the biopsy tool near the first end thereof. Alternatively, the user can secure the biopsy tool within a speculum or other similar device for use in moving the device. The user can press the cutting blade 20 on the second end 14 of the elongated shaft 12 into the tissue of a user in a twisting motion.

The loop 17 of the cutting wire 16 is positioned adjacent to the interior of the cutting blade 20 and is positioned around the tissue sample. In order to sever the base or underside of the tissue sample to allow the sample to be removed from the user, the user can pull the cutting wire 16. In a first position, the loop 17 comprises a large diameter, similar to that of the cutting blade 20. By pulling the cutting wire 16 at the first end of the elongated shaft 12, the diameter of the loop 17 decreases, cutting the underside of the tissue sample. As the cutting wire 16 is pulled, the loop cuts the base or underside of the tissue sample. The tissue sample is then held within the second end 14 of the elongated shaft 12 within the cutting blade 20.

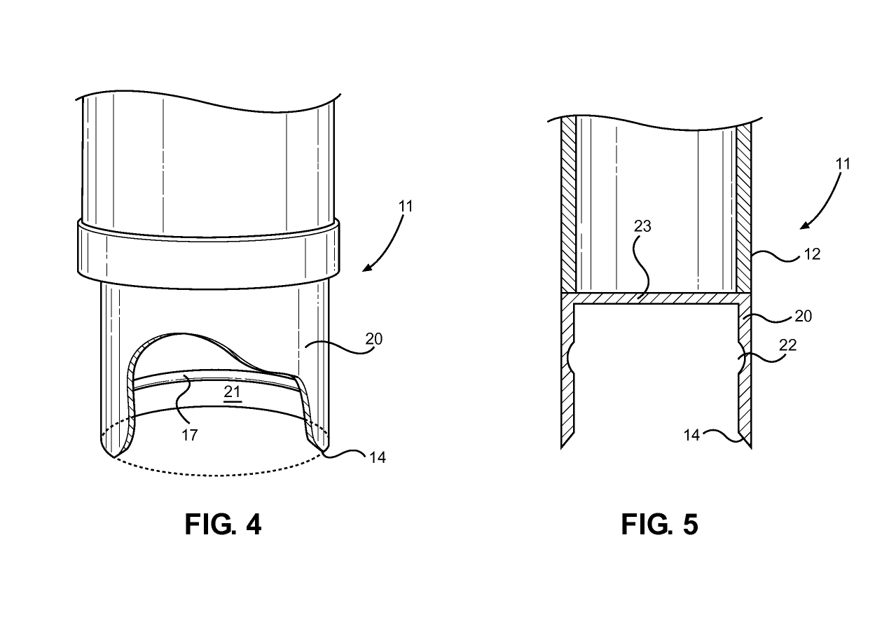

Referring now to FIGS. 4 and 5, there are shown views of the cutting blade of the biopsy tool. In a preferred embodiment, the cutting wire 17 is removably affixed to the interior surface 21 of the cutting blade 20. In this way, the cutting wire 17 remains in position against the cutting blade while the cutting blade 20 is inserted into tissue. The cutting wire 17 can then be released from the cutting blade 20 when the user pulls the end of the cutting wire 17 so as to sever the base of the tissue sample.

In some embodiments, the interior surface 21 of the cutting blade 20 comprises an adhesive thereon that helps to temporarily secure the cutting wire 17 thereto. In alternate embodiments, as shown in FIG. 5, the interior surface comprises a channel 22 in which the cutting wire 17 is removably positioned. The cutting wire 17 is temporarily affixed within the channel 22 until the cutting wire 17 is pulled, wherein the cutting wire 17 is freed from the channel 22 so as to cut into tissue. The channel 22 circumscribes the interior surface of the cutting blade 22 so as to form a circular or loop configuration.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A biopsy tool, comprising:
an elongated shaft having a hollow interior volume, an open first end, and an open second end;
a cutting blade disposed on the second end;
a cutting wire extending through the hollow interior volume and forming a loop at the second end of the elongated shaft, wherein the cutting wire is movable by a user in order to reduce the size of the loop so that the loop cuts a tissue of a user freeing the tissue from the user.

2) The biopsy tool of claim 1, wherein the elongated shaft is cylindrical in configuration.

3) The biopsy tool of claim 1, wherein the elongated shaft comprises a threaded exterior adapted to removably engage with a speculum.

4) The biopsy tool of claim 1, wherein the cutting wire extends from the first end of the elongated shaft.

5) The biopsy tool of claim 1, further comprising a support cuff disposed within the interior volume of the elongated shaft, wherein the cutting wire extends through the support cuff such that the support cuff restrains movement of the cutting wire.

6) The biopsy tool of claim 1, wherein the loop is adapted to cut tissue disposed within the interior area of the loop when a portion of the cutting wire adjacent to the first end of the elongated shaft is pulled.

7) The biopsy tool of claim 1, wherein the cutting blade comprises a circular configuration.

8) The biopsy tool of claim 1, wherein the cutting blade comprises a channel on an interior surface thereof, wherein the cutting wire is removably positioned within the channel.
